(19) United States
(12) Patent Application Publication
(10) Pub. No.: US 2022/0375552 A1
(43) Pub. Date: Nov. 24, 2022

(54) Secure Intelligent Networked Architecture

(71) Applicant: Intellihealth Inc., San Francisco, CA (US)

(72) Inventors: Katherine Saunders, New York, NY (US); Louis Aronne, Greenwich, CT (US); Sloan Saunders, New York, NY (US); David H. Bass, San Francisco, CA (US); Susan Campbell Weir, Clayton, CA (US); Leon Igel, New York, NY (US)

(21) Appl. No.: 17/327,420

(22) Filed: May 21, 2021

(51) Int. Cl. G16H 10/60; G06F 21/71; G06F 21/62; G16H 20/00

(52) U.S. Cl. CPC G16H 10/60; G06F 21/71; G06F 21/6245; G16H 20/00

(57) ABSTRACT

According to some exemplary embodiments, a secure intelligent networked architecture is provided comprising a secure intelligent variable determining agent, the intelligent variable determining agent configured to automatically determine a first plurality of digital data elements, a secure intelligent data agent, the secure intelligent data agent having secure digital data corresponding to the first plurality of digital data elements, a secure intelligent insight agent configured to receive from the secure intelligent data agent the secure digital data corresponding to the first plurality of digital data elements and configured to transform the secure digital data corresponding to the first plurality of digital data elements into a scrubbed trigger, the scrubbed trigger being a reduced size version of the secure digital data, and a secure intelligent action agent configured to receive the scrubbed trigger and cause a first action.

### SUMMARY OF EXEMPLARY EMBODIMENTS

According to some exemplary embodiments, a secure intelligent networked architecture is provided comprising a secure intelligent variable determining agent, the intelligent variable determining agent configured to automatically determine a first plurality of digital data elements (for example, determining variables to query such as a person's gender, medications, current health conditions, past health conditions, answers to a questionnaire, body mass index (“BMI”), etc.), a secure intelligent data agent, the secure intelligent data agent having secure digital data corresponding to the first plurality of digital data elements (for example, medical records, info received from clinics, patient devices, patients corresponding to the first plurality of digital data elements), a secure intelligent insight agent configured to receive from the secure intelligent data agent the secure digital data corresponding to the first plurality of digital data elements and configured to transform the secure digital data corresponding to the first plurality of digital data elements into a scrubbed trigger, the scrubbed trigger being a reduced size version of the secure digital data (for example, receiving a vast amount of data, identifying the key data, reducing the required memory and increasing processing speed), and a secure intelligent action agent configured to receive the scrubbed trigger and cause a first action (for example, the taking of a medication, following a nutritional regimen, exercising, etc.).

In further exemplary embodiments, the secure intelligent networked architecture includes the first action causing a first change in a first performance metric (for example, BMI, weight, blood pressure, glucose level, etc.), the first performance metric being securely transmitted by a networked device (for example, from the internet of things (“TOT”) an encrypted transmission) to the secure intelligent networked architecture and automatically adjusting the first action, automatically resulting in a second action based on the transmitted first performance metric received by the secure intelligent networked architecture (for example, making the system smarter). The second action may cause a second change in the first performance metric, automatically resulting in a second performance metric (for example, the system is dynamic). The second performance metric may be securely transmitted by the networked device to the secure intelligent networked architecture. The secure intelligent networked architecture may automatically adjust the second action automatically resulting in a third action based on the transmitted second performance metric received by the secure intelligent networked architecture.

The secure intelligent networked architecture, according to many exemplary embodiments, may correlate each of the first plurality of digital data elements to each of the performance metrics, correlate each of the secure digital data corresponding to each of the first plurality of digital data elements to each of the performance metrics, and/or correlate the each of the secure digital data corresponding to each of the first plurality of digital data elements to each of the performance metrics to each scrubbed trigger.

The secure intelligent networked architecture, in various exemplary embodiments, may include the secure intelligent variable determining agent configured to utilize a random number generator machine to select the first plurality of digital data elements. Additionally, a load balancer may be configured to distribute the secure digital data received by the secure intelligent insight agent and any additional secure intelligent insight agents. The load balancer may be further configured to distribute processing of the secure digital data received by the secure intelligent insight agent and any additional secure intelligent insight agents.

In yet further exemplary embodiments, the secure intelligent insight agent is configured to perform destruction of secure digital data to increase a speed of each subsequent processing run. A plurality of hardware based secure, high data transfer corridors, each having specialized processors and switches may facilitate unilateral or bilateral transfer of the secure digital data between any of the secure intelligent variable determining agent, the secure intelligent data agent, the secure intelligent insight agent and the secure intelligent action agent.

The secure intelligent networked architecture, in certain exemplary embodiments, may predict a human phenotype based on some or all of data in the intelligent networked architecture. Additionally, a plurality of actions may cause changes in a plurality of performance metrics, the plurality of performance metrics statistically associated with an improvement in a primary chronic condition. The plurality of performance metrics may be statistically associated with an improvement in a secondary chronic condition and/or with an improvement in a tertiary chronic condition.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 shows an exemplary secure intelligent networked architecture.

FIGS. 2A-2C show an exemplary method for a secure intelligent networked architecture.

Appendix A shows an exemplary patient questionnaire.

Appendix B shows an exemplary medication decision support recommendation algorithm.

Appendix C shows exemplary provider and patient health information cards that are the images for the proprietary health risk assessment questionnaires (e.g. in Appendix A which create the algorithm answers and output to the exemplary provider and patient recommendation cards).

Appendix D shows exemplary trend support algorithms.

### DETAILED DESCRIPTION

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration, but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In the following description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted at the outset that the terms “coupled,” “connected”, “connecting,” “electrically connected,” etc., are used interchangeably herein to generally refer to the condition of being electrically/electronically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various figures (including component diagrams) shown and discussed herein are for illustrative purpose only, and are not drawn to scale.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel, or may be performed at different times.

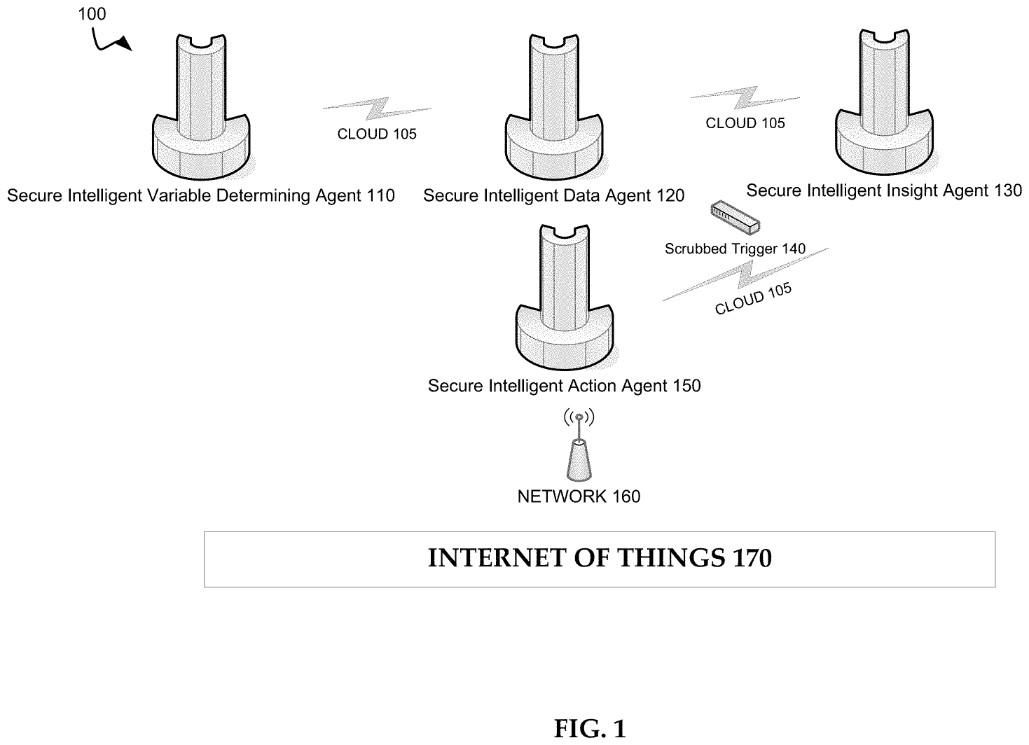

FIG. 1 is a diagram of an exemplary system 100 for secure intelligent networked architecture, processing and execution.

The exemplary system 100 as shown in FIG. 1 includes a secure intelligent variable determining agent 110, secure intelligent data agent 120, secure intelligent insight agent 130, secure intelligent action agent 150, network 160 and internet of things 170. Also shown in FIG. 1 are clouds 105, and scrubbed trigger 140.

According to various exemplary embodiments, the secure intelligent variable determining agent 110 is a non-generic computing device comprising non-generic computing components. It may comprise specialized dedicated processors configured to automatically determine a first plurality of digital data elements.

The secure intelligent data agent 120, in various exemplary embodiments, includes a specialized hardware processor and a memory having secure digital data corresponding to the first plurality of digital data elements.

The secure intelligent insight agent 130 has a specialized hardware processor and a memory configured to receive from the secure intelligent data agent 120 the secure digital data corresponding to the first plurality of digital data elements and is configured to transform the secure digital data corresponding to the first plurality of digital data elements into a scrubbed trigger 140. The scrubbed trigger 140 is a reduced size version of the secure digital data.

The secure intelligent action agent 150 has a specialized hardware processor and a memory and is configured to receive the scrubbed trigger 140 and cause a first action. The first action may cause a first change in a first performance metric. The first performance metric may be securely transmitted by a networked device included in the internet of things (“IOT”) over the network 160 to the secure intelligent networked architecture 100.

In some exemplary embodiments, the secure intelligent variable determining agent 110, the secure intelligent data agent 120, the secure intelligent insight agent 130 and/or the secure intelligent action agent 150 is situated behind a firewall (not shown). In some embodiments, the secure intelligent data agent 120 encrypts the secure digital data before storing it and decrypts the secure digital data before copying and transmitting it to the secure intelligent insight agent 130 or to the secure intelligent action agent 150. The decrypting may be performed as a separate step in advance of copying and/or transmitting to increase the speed in which the specialized dedicated processors may copy and/or transmit the secure digital data.

The secure intelligent variable determining agent 110, in some exemplary embodiments, is configured to utilize a random number generator machine to select the first and/or subsequent plurality of digital data elements. Additionally, the intelligent networked architecture may comprise a load balancer having a specialized hardware processor, and the load balancer is configured to distribute the secure digital data received by the secure intelligent insight agent 130 and any additional secure intelligent insight agents. Furthermore, the load balancer may be configured to distribute processing of the secure digital data received by the secure intelligent insight agent 130 and any additional secure intelligent insight agents.

The secure intelligent insight agent 130 may be configured to perform destruction of secure digital data to increase a speed of each subsequent processing run. A plurality of hardware based secure, high data transfer corridors, each having specialized processors and switches may facilitate unilateral or bilateral transfer of the secure digital data between any of the secure intelligent variable determining agent 110, the secure intelligent data agent 120, the secure intelligent insight agent 130 and/or the secure intelligent action agent 150.

In further exemplary embodiments, the secure intelligent variable determining agent 110, the secure intelligent data agent 120, the secure intelligent insight agent 130 and/or the secure intelligent action agent 150 may employ artificial intelligence and machine learning. Numerous determination steps by the secure intelligent variable determining agent 110 as described herein may be made by an automatic machine determination without human involvement, including being based on a previous outcome or feedback (e.g. an automatic feedback loop) provided by the secure intelligent networked architecture, processing and/or execution as described herein.

The optional hardware based random number generator machine (not shown), according to various exemplary embodiments, may determine and/or transmit one or more digital data elements to the secure intelligent variable determining agent 110, the secure intelligent data agent 120, the secure intelligent insight agent 130 and/or the secure intelligent action agent 150.

In certain exemplary embodiments, the secure intelligent networked architecture 100 may include a virtual machine (not shown). A virtual machine may comprise an emulation of a particular computer system. Virtual machines operate based on the computer architecture and functions of a real or hypothetical computer, and their implementations may involve specialized hardware, software, or a combination of both.

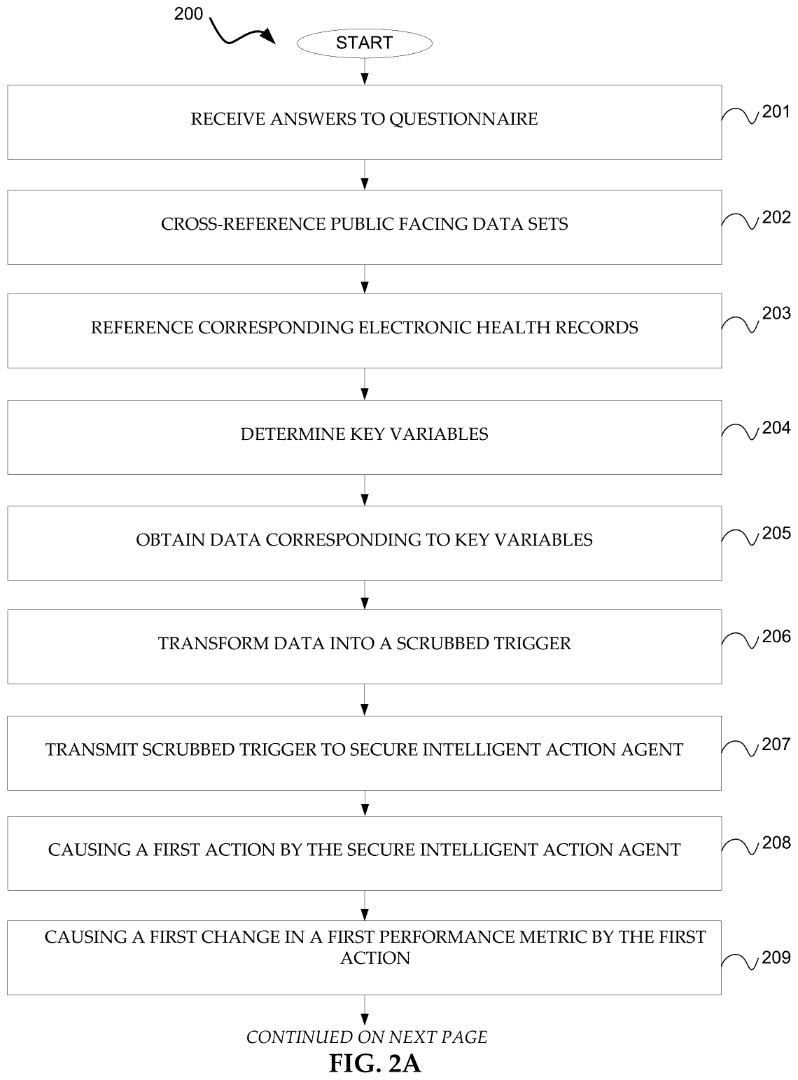

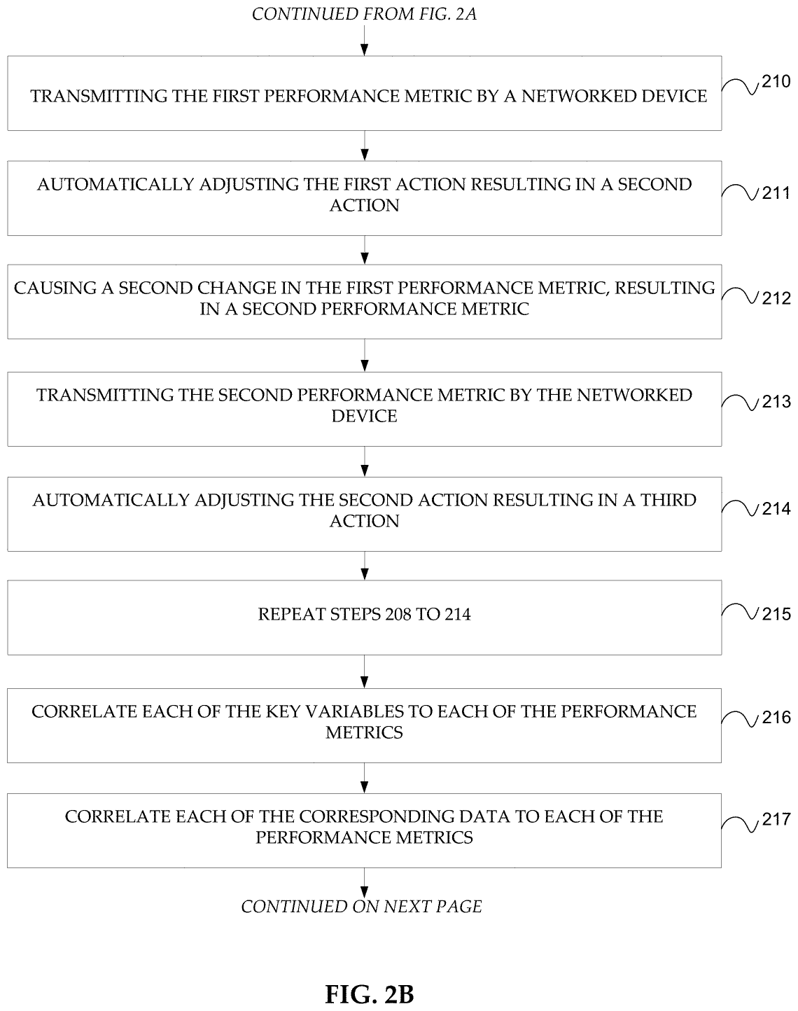

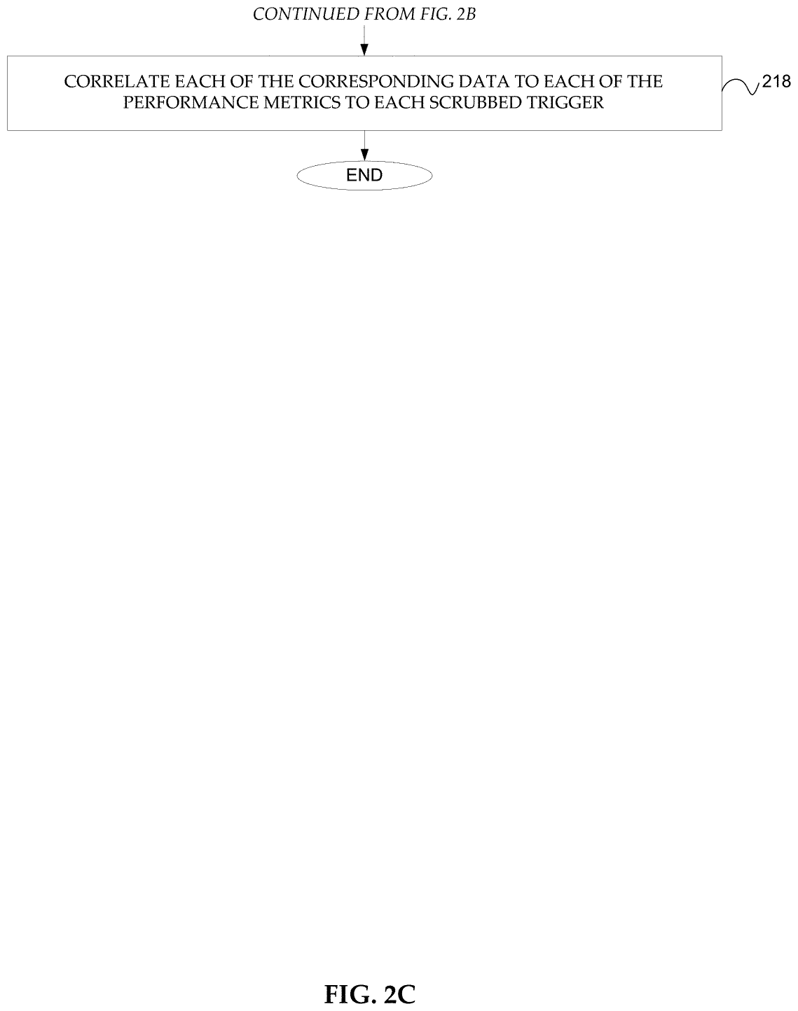

FIGS. 2A-2C show an exemplary method 200 for a secure intelligent networked architecture.

At step 201, the secure intelligent data agent 120 (FIG. 1) receives answers to a questionnaire. In some exemplary embodiments, the questions may be found in Appendix A herein.

Appendix A comprises a series of questions on topics such as weight, medical conditions, nutrition, fitness, sleep, lifestyle, etc. In addition to receiving answers from a patient, the questionnaire may receive input from an electronic health record. In some embodiments, the questionnaire may receive information from public facing databases such as medication side-effects or disease related information. Additionally, as the patient inputs information, the system may offer suggestions and/or offer a pull-down menu of selections or the like.

Upon receiving the information, various algorithms will be applied to the information. As shown in Appendix A, two sets of health information cards, one for the provider and one for the patient will be generated. Next to each card are placement locations for where various pieces of information should automatically be placed. The provider and patient health information cards are shown in greater detail in Appendix C.

In further exemplary embodiments, the secure intelligent data agent 120 may receive genetic sequence information for a particular patient and/or user of the system.

At step 202, the secure intelligent data agent 120 may cross-reference over a network public facing data sets based on the received information at step 201. For example, if a patient reports taking a particular medication, FDA databases may be cross-referenced for side effects associated with that medication.

At step 203, the secure intelligent data agent 120 may reference over a network an electronic health record (“EHR”) corresponding to the particular patient addressed at steps 201-202. In some cases, the electronic health record may reside in the secure intelligent data agent 120.

At step 204, the secure intelligent variable determining agent 110 (FIG. 1) may determine key variables for a patient. Exemplary variables may include a patient's gender, medication(s), a current health condition, a past health condition, an answer to a questionnaire, body mass index (“BMI”), etc.

At step 205, data corresponding to the key variables may be obtained from the secure intelligent data agent 120. Such data may include a medical record, data received from a clinic, a patient device, directly from a patient, etc. that corresponds to the first plurality of digital data elements determined at step 204.

At step 206, the secure intelligent insight agent 130 (FIG. 1), receives from the secure intelligent data agent 120 the secure digital data corresponding to the first plurality of digital data elements determined at step 204 and transforms the secure digital data corresponding to the first plurality of digital data elements into a scrubbed trigger, the scrubbed trigger being a reduced size version of the secure digital data, which reduces the required computer memory and increases the speed of data processing.

At step 207, the scrubbed trigger is transmitted to the secure intelligent action agent 150 (FIG. 1).

At step 208, a first action is caused by the secure intelligent action agent 150. Such actions may include the taking of a medication, adopting a particular nutritional regimen, performing an exercise, etc. For example, Appendix B shows an exemplary medication decision support recommendation algorithm that may be used for recommending an action with respect to the taking of a medication.

As shown in Appendix B, a rules engine generates various variables that should be applied to make a medication recommendation. For example, check diagnosis, medication, gender, use of stimulants, Monoamine oxidase inhibitors (MAOIs), antiretrovirals and antiepileptics. Accordingly, a medication will be recommended provided certain patient conditions are satisfied. In exemplary embodiments, patient responses (e.g. changes in the various variables) may be received by the rules engine and based on such feedback, future medication recommendations may be automatically adjusted for a patient and/or a group of patients.

In some embodiments, the recommended medication will be shown on the patient and/or provider health information card as shown in Appendix C.

Appendix C shows exemplary provider and patient health information cards. The provider health information card shows clinical recommendations. The patient health information card includes recommendations on such issues as weight, nutrition, sleep, lifestyle, medical conditions, exercise and eating.

At step 209, the first action causes a first change in a first performance metric. For example a first change in a first performance metric may include a change in a body mass index (“BMI”), weight, blood pressure, behavior, or in a glucose level, etc.

At step 210, the first performance metric is transmitted from a device belonging to an internet of things 170 (FIG. 1) over a network 160 (FIG. 1) to the secure intelligent action agent 150.

At Step 211, the first action is automatically adjusted by the secure intelligent action agent 150, resulting in a second action based on the transmitted first performance metric received by the secure intelligent action agent 150, making the secure intelligent network architecture smarter.

At step 212, a second change in the first performance metric is caused, resulting in a second performance metric.

At step 213, the second performance metric is transmitted from a device belonging to an internet of things 170 over a network 160 to the secure intelligent action agent 150.

At step 214, the second action is automatically adjusted by the secure intelligent action agent 150, resulting in a third action based on the transmitted second performance metric received by the secure intelligent action agent 150, making the secure intelligent network architecture even smarter.

At step 215, step 208-214 are repeated.

At step 216, each of the key variables are correlated to each of the performance metrics.

At step 217, each of the corresponding data is correlated to each of the performance metric.

At step 218, each of the corresponding data to each of the performance metrics is correlated to each scrubbed trigger. Additionally, Appendix D shows exemplary trend support algorithms.

As shown in Appendix D, the system is continuously automatically evaluating such factors as the relationship between age, gender, time a patient spends interacting with the system, medication and weight loss. This is just one evaluation out of approximately 130 evaluations. Advantageously, data points may be generated that are behavioral. For example, a 45 year old woman with a body mass index (“BMI”) at a certain level, or is trending and gaining, smart tools may alert providers that they need to engage with this person.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A secure intelligent networked architecture comprising:
a secure intelligent variable determining agent having a specialized hardware processor and a memory, the intelligent variable determining agent configured to automatically determine a first plurality of digital data elements;
a secure intelligent data agent, the secure intelligent data agent having a specialized hardware processor and a memory further comprising secure digital data corresponding to the first plurality of digital data elements;
a secure intelligent insight agent, the secure intelligent insight agent having a specialized hardware processor and a memory, and configured to receive from the secure intelligent data agent the secure digital data corresponding to the first plurality of digital data elements and configured to transform the secure digital data corresponding to the first plurality of digital data elements into a scrubbed trigger, the scrubbed trigger being a reduced size version of the secure digital data; and
a secure intelligent action agent having a specialized hardware processor and a memory, the secure intelligent action agent configured to receive the scrubbed trigger and cause a first action.

2. The secure intelligent networked architecture of claim 1, further comprising the first action causing a first change in a first performance metric.

3. The secure intelligent networked architecture of claim 2, further comprising the first performance metric being securely transmitted by a networked device to the secure intelligent networked architecture.

4. The secure intelligent networked architecture of claim 3, further comprising automatically adjusting the first action automatically resulting in a second action based on the transmitted first performance metric received by the secure intelligent networked architecture.

5. The secure intelligent networked architecture of claim 4, further comprising the second action causing a second change in the first performance metric, automatically resulting in a second performance metric.

6. The secure networked architecture of claim 5, further comprising the second performance metric being securely transmitted by the networked device to the secure intelligent networked architecture.

7. The secure intelligent networked architecture of claim 6, further comprising automatically adjusting the second action automatically resulting in a third action based on the transmitted second performance metric received by the secure intelligent networked architecture.

8. The secure intelligent networked architecture of claim 7, further comprising correlating each of the first plurality of digital data elements to each of the performance metrics.

9. The secure intelligent networked architecture of claim 8, further comprising correlating each of the secure digital data corresponding to each of the first plurality of digital data elements to each of the performance metrics.

10. The secure intelligent networked architecture of claim 9, further comprising correlating the each of the secure digital data corresponding to each of the first plurality of digital data elements to each of the performance metrics to each scrubbed trigger.

11. The secure intelligent networked architecture of claim 1, further comprising the secure intelligent variable determining agent configured to utilize a random number generator machine to select the first plurality of digital data elements.

12. The intelligent networked architecture of claim 1, further comprising a load balancer having a specialized hardware processor, the load balancer configured to distribute the secure digital data received by the secure intelligent insight agent and any additional secure intelligent insight agents.

13. The intelligent networked architecture of claim 12, wherein the load balancer is further configured to distribute processing of the secure digital data received by the secure intelligent insight agent and any additional secure intelligent insight agents.

14. The intelligent networked architecture of claim 13, wherein the secure intelligent insight agent is configured to perform destruction of secure digital data to increase a speed of each subsequent processing run.

15. The intelligent networked architecture of claim 1, further comprising a plurality of hardware based secure, high data transfer corridors, each having specialized processors and switches to facilitate unilateral or bilateral transfer of the secure digital data between any of the secure intelligent variable determining agent, the secure intelligent data agent, the secure intelligent insight agent and the secure intelligent action agent.

16. The secure intelligent networked architecture of claim 1, further comprising predicting a human phenotype based on some or all of data in the intelligent networked architecture.

17. The secure intelligent networked architecture of claim 1, further comprising a plurality of actions causing changes in a plurality of performance metrics, the plurality of performance metrics statistically associated with an improvement in a primary chronic condition.

18. The secure intelligent networked architecture of claim 17, further comprising the plurality of performance metrics statistically associated with an improvement in a secondary chronic condition.

19. The secure intelligent networked architecture of claim 18, further comprising the plurality of performance metrics statistically associated with an improvement in a tertiary chronic condition.
